(19) United States
(12) Patent Application Publication
(10) Pub. No.: US 2007/0178046 A1
(43) Pub. Date: Aug. 2, 2007

(54) System for correlating odors and tastes to objective elements

(76) Inventors: Rachel S. Herz, Warwick, RI (US); Kathleen A. McCann, Riverside, RI (US)

Correspondence Address: MOORE & HANSEN, PLLP, 225 SOUTH SIXTH ST, MINNEAPOLIS, MN, 55402, US

(21) Appl. No.: 11/634,509

(22) Filed: Dec. 5, 2006

Related U.S. Application Data
(60) Provisional application No. 60/742,744, filed on Dec. 5, 2005.

(51) Int. Cl. A61K 49/00

(52) U.S. Cl. 424/9.1

(57) ABSTRACT

A kit containing a plurality of scent/flavor samples and taste samples contained in individual vessels. Methods of teaching an understanding of taste, smelling with the nose, smelling with the mouth, and the concept of “flavor,” is facilitated. Numerous possibilities of combining scents with tastes, thus enabling a person to explore creatively and deductively the creation of new scent and flavor mixtures, as well as learning what makes familiar flavors taste the way they do.

### RELATIONSHIP TO OTHER APPLICATION

This application claims the benefit of the filing date of U.S. Provisional Patent Application Ser. No. 60/742,744 filed on Dec. 5, 2005, the disclosure of which is incorporated herein by reference.

### BACKGROUND OF THE INVENTION

1. Field of the Invention

This invention relates generally to systems for correlating perception to corresponding descriptive language, and more particularly, to a system that facilitates the systematic teaching of appropriate language that identifies and describes corresponding olfactory and flavor experiences.

2. Description of the Related Art There is a significant disconnect between language and odor perception. People have more difficulty coming up with words to describe their scent experiences than they have for any other sensation. Moreover, when words are applied to olfactory experience they are often random and idiosyncratic. This is because children are not taught the connection between scents and their names (semantic representation) in any systematic way, as they are with other sensory experiences. Indeed, our varied personal experiences with scents, and hence what we call them, has been a primary obstacle in the psychological understanding of odor perception for decades. The present invention constitutes the first step toward breaking this trend and providing children with the unique opportunity to learn specific conceptual and linguistic relationships to a varied and wide set of specific odorants.

Taste and smell are very different psychological and perceptual senses. However, because we receive so little formal training about these senses, most people don't understand the differences between taste and smell, and are not aware that the sense of smell is essential for our experience of flavor. This invention is the first educational and play tool to teach children how taste comprises only the four basic sensations of: salt, sour, sweet and bitter and that everything else that we experience in our mouths as flavor comes from smell. Through learning about flavor, children will discover that smells can be perceived both in the mouth and through the nose. The only difference between the ‘taste’ of a licorice jelly-bean and a lime jelly-bean are the aromas that have been added to the sweet sugar based jelly. This is why when you have a cold or you nose is plugged, things do not taste “right.” The airflow from the mouth to the nose needed for flavor/scent perception is obstructed. A demonstration of this phenomenon can be played with using the present invention and plugging one's nose.

Mixtures of tastes and mixtures of smells possess different qualities, and mixtures of both tastes and smells are also in their own category of perceptual experience. A mix of salty and sour tastes produces a taste sensation that is at once both salty and sour (analytic sensation), whereas a mix of rose and cheddar cheese scents produces a new scent that is holistic and different (synthetic sensation). Mixtures of smells and tastes together produce flavors that have both synthetic and analytic components. The present invention will enable children to explore and experiment with taste and smell mixture creation and to deductively discover how familiar (and unfamiliar) flavors are developed (e.g., the taste of sweet and the scent of orange produces the familiar orange juice flavor, but the taste of salty and the scent of orange does not).

Starting at age 6, and up to about age 12, children are in what has been called the “concrete operational stage.” In this important developmental stage, children are capable of a variety of mental transformations and operations. They are also very creative and eager to explore their world and engage in creative play. Another key factor is that children at this stage are particularly tied to the physical world around them. Therefore, exposure to sensory experiences (smell and taste) and combining them with language concepts is both very appealing to children and highly beneficial.

The challenges offered by playing with the present invention will build upon a child's abilities and talents, produce greater cognitive and psychological flexibility, and increase children's intelligence and creativity.

There is currently no existent product (tool, kit or toy) in educational or play format that provides a hands-on, interactive, creative experience for teaching children about the senses of smell and taste. The present invention, through both direct and deductive learning, is the first ever item that will increase children's sensory intelligence for smell and taste.

### SUMMARY OF THE INVENTION

The foregoing and other objects are achieved by this invention which provides, in accordance with a first method aspect of the invention, a method of educating a living being to correlate taste and olfactory perception to an objective element. In one embodiment, the method includes the steps of:

providing a plurality of known taste ingestibles;

providing a plurality of known flavor ingestibles;

combining selectable ones of the flavor ingestibles and taste ingestibles to form a combination ingestible;

causing the living being to ingest the combination ingestible; and

training the living being to identify the combination ingestible by reference to the objective element.

In embodiments where the living being is a human being, and the objective element is a word that characterizes the perception resulting from the ingestion of the combination ingestible. Of course, other objective elements, such as colors, may be associated with the perception resulting from the ingestion of the combination ingestible.

The combination ingestible may be formed of a combination exclusively of a plurality of the known taste ingestibles, a combination exclusively of a plurality of the known flavor ingestibles, or combinations of both.

In embodiments where the living being is an animal, the objective element is a predetermined scent. For example, the predetermined scent is in some embodiments a contraband scent.

The plurality of known taste ingestibles is selected, in some embodiments, from the group of salt, sour, sweet, and bitter. Moreover, the plurality of known flavor ingestibles is selected from the group of cinnamon, orange, licorice, chocolate, green-pepper, cheddar cheese, spearmint, and coconut.

In a highly advantageous embodiment of the invention, the method of educating a living being to correlate taste and olfactory perception to an objective element is configured as a guessing game. In such a game, the plurality of known taste ingestibles and the plurality of known flavor ingestibles are configured to be unidentifiable. The known taste ingestibles and the plurality of known flavor ingestibles are, in some embodiments, liquids that have been configured by color and/or viscosity not to be representative of any taste or scent.

In accordance with a further method aspect of the invention, there are provided the steps of:

combining selectable ones of a plurality of ingestibles to produce a combination ingestible;

ingesting the combination ingestible; and

guessing a word that characterizes the perception resulting from the step of ingesting.

In one embodiment of this further method aspect of the invention, the step of combining selectable ones of a plurality of ingestibles includes the step of combining selectable ones of a plurality of known taste ingestibles. The known taste ingestibles are selected from the group of salt, sour, sweet, and bitter. In other embodiments, the step of combining selectable ones of a plurality of ingestibles includes the step of combining selectable ones of a plurality of known flavor ingestibles. The known flavor ingestibles are selected from the group of cinnamon, orange, licorice, chocolate, green-pepper, cheddar cheese, spearmint, and coconut. The step of combining selectable ones of a plurality of ingestibles includes, in some embodiments, the step of combining selectable ones of a plurality of known taste ingestibles and a plurality of known flavor ingestibles.

In accordance with an apparatus aspect of the invention, there are provided a plurality of known taste ingestibles and a plurality of known flavor ingestibles. Additionally, there is provided an edible substrate for mixing selectable ones of the known taste ingestibles and the known flavor ingestibles.

In one embodiment of this apparatus aspect of the invention, there is further provided a rotatable carrier for supporting the plurality of known taste ingestibles and the plurality of known flavor ingestibles rotatively. In other embodiments, there are further provided a blindfold, a nose clip, and a plurality of selectable cards containing instructions for a player.

In a further aspect of the invention, there is provided a kit containing a plurality of scent samples and taste samples contained in individual vessels.

In a specific illustrative embodiment of the invention the invention may be in the form of a kit, a toy, or a game, that contains eight scent samples. Of course, many other scent samples may be used in the practice of the invention, as will be noted hereinbelow. Additionally, there are included four tastants (salt, sour, sweet, bitter) in 15 ml (size can vary) glass/plastic/paper/metal dispensers. Scents and tastes are hereafter referred to as “samples.” Sample dispensers may, in various embodiments of the invention, include lids, flip-tops, or other forms of seals. All samples are edible. Disbursement of samples are in any of the following form: liquid spray, liquid drops, film, gel, or (micro)encapsulated beads. Samples may be clear or colored. Dispensers are monochrome and labeled with any or all of the following: coded number, name, picture (illustration/photo) of the source. In the prototype, dispensers are monochrome white (color can vary). Sample names in black lettering are displayed vertically on the side of the dispensers. A colored picture of the sample source are on the side and/or top cover of the dispenser and coded numbers may be on the bottom of the dispensers. An edible and scent/taste neutral medium (i.e. wafer or rice paper) may be included with the invention to absorb the samples if in liquid or gel form.

The scent and taste dispensers are contained in a hard paper/plastic/glass/metal box. The prototype box, in this embodiment, is monochrome (color may vary) plastic with a clip/snap/buckle closing lid of dimensions measuring approximately 7 inches wide, 5 inches deep, 4 inches tall. Other sizes of enclosures can be used in the practice of the invention. Inside the enclosure are provided in a practical embodiment of the invention twelve wells for the scent and taste dispensers. In one embodiment, they are arranged in three rows of four samples each. Two of the rows are for scents and one row is for tastes. The wheels that accommodate the samples are vertical, flat, or tiered. Moreover, the inside lid of the box will map out where each scent and taste is located with pictures to ensure consistent replacement in the designated well. This will minimize odor/taste contamination. It is desired that contamination between odorants and tastes be minimized during use of the invention.

The invention may include a workbook for recording scent/taste/flavor combinations and experiments, and a blindfold for enhanced sensory experience and game playing. Also included are instructions and suggestions for ways to use the invention and a code key for samples with code number labeling.

Allergic reactions to the natural chemicals used in the making of the odorants and tastants in this invention are rare, however, appropriate warnings are affixed to all packaging. Similarly, though not damaging, the chemicals used may be eye irritants and as warnings not to spray or apply into eyes also are affixed to packaging. No components to which there are already known allergic reactions, such as peanuts, are intended to be used.

There is minimal risk of contamination from sticky fingers or transfer of illness between children playing with the invention. The odorants and tastants are all be food grade edible with the same risk level as flavored candy. Superior to health issues related to ingesting candy, the odorants and tastants in this invention are of negligible to no calorie content.

There are numerous applications of the invention. These include, for example:
Education: An educational tool to be used in classroom as well as nontraditional educational settings by teachers engaged in a curriculum pertinent to teaching about the senses of smell and taste.
Toys: A fun and educational toy most directed, but not limited to, children between the ages of 6-12.
Games: The instructions enclosed with the invention and materials included with it can be modified for use in various game strategies, including but not limited to: board games, guessing games, card games.
Specific scents, graphics, instructional material and packaging design may vary as a function of primary usage.

The present invention provides a first step toward ending the poor verbal communication abilities most have for smell experiences. It provides to children an opportunity to learn specific conceptual and linguistic relationships between smells and words by directly connecting familiar scents and tastes with their prototypical names along with colorful illustrations of their source. In addition, the present invention will facilitate an understanding of taste, smelling with the nose, smelling with your mouth, and the concept of “flavor:” Children in particular will learn that taste comprises only the four basic sensations of: salt, sour, sweet and bitter and that everything else that is experienced as flavor is derived from smell. By playing and experimenting with the tastes and scents both orally and through their noses children will come to understand the different ways that aromas are perceived (mouth and nose), the differences between the senses of smell and taste, and how flavors are created.

In addition to the foregoing, the present invention teaches multisensory awareness, analytic and creative play, as well motor enhancement, by combining the sensory experiences of smell and taste with visual images that will foster a child's understanding of how perceptual experiences can be represented in multiple sensory forms. This invention allows for numerous possibilities of combining scents with tastes, thus enabling a student to explore creatively and deductively the creation of new scent and flavor mixtures, as well as learning what makes familiar flavors taste the way they do. Creative play and exploration with this invention will enhance a child's deductive and analytic skills as well as their cognitive and perceptual flexibility, and will enrich their sensory awareness and sensory intelligence. The manipulation of the scent and taste dispensers will advance physical and motor skills by enhancing dexterity and nurturing physical-intellectual development.

Certain embodiments of the invention are directed, but not limited, to children between the ages of 6-12. Starting at age 6, and up to about age 12, children are in the concrete operational stage of development. Intellectual gains and benefits incurred during this stage have the greatest possibility for increasing a child's intellectual abilities. Sensory experiences are very salient during this stage of development, and exposure to multiple sensations (smell, taste, and sight) and combining them with language concepts is both very appealing to children and highly beneficial. This invention can be modified for use with pre-school age children, as well as for use with adults wishing to learn and experiment with culinary and perfume scent and flavor creation.

Illustrative tastants and scents to be used in the practice of a specific illustrative embodiment of the invention include:
Salt Sour Sweet Bitter

The four tastants in this embodiment are a constant in all the variations of this invention that include a taste and flavor component. It is to be noted that variations of the present invention may involve scents only. Scents will vary depending upon need and are obtained from any number of categories including: flowers, fruits, spices, outdoorsy scents and condiments. It is not necessary that the scents be familiar or pleasant. The following are illustrative of some of the scents that can be used in the practice of the invention:

| | | |
|---|---|---|
| Coconut | Garlic | Rose |
| Lavender | Vinegar | Grape |
| Chocolate | Mustard | Popcorn |
| Orange | Peanut butter | Bell Pepper |
| | (not containing | |
| | real peanuts) are | |
| Licorice | Almond | Caraway |
| Peppermint | Maple | Coriander |
| Cedar wood | Caramel | Rosemary |
| Fresh Cut Grass | Cheddar Cheese | Cardamom |
| Hay | Cinnamon | Clove |
| Seaweed | Lilac | Bergamot |
| Strawberry | Carrot | Verbena |

The invention is an educational tool, game or toy that teaches children about the senses of smell and taste in a hands-on, interactive and creative manner. In particular, language labels and visual concepts will for the first time be specifically attached to various scents and tastes for prototype learning. The invention contains easy and fun to manipulate scent and taste dispensers with scent and taste samples delivered in a reliable, safe and edible format. Instructional materials and additional items, such as a blindfold, may be including in packaging of the invention. This invention is the first ever product to expand and enrich sensory intelligence for smell and taste.

### BRIEF DESCRIPTION OF THE DRAWING

Comprehension of the invention is facilitated by reading the following detailed description, in conjunction with the annexed drawing, in which:

FIG. 1 is a simplified representation of a specific illustrative embodiment of the invention configured as a kit having three rows of four samples;

FIG. 2 is an enlarged representation of a fragrance sample container as shown in FIG. 1, in open and closed conditions; and

FIG. 3 is a perspective representation of a further specific illustrative embodiment of the invention, configured as a kit having a plurality of samples in a rotatable game arrangement.

### DETAILED DESCRIPTION

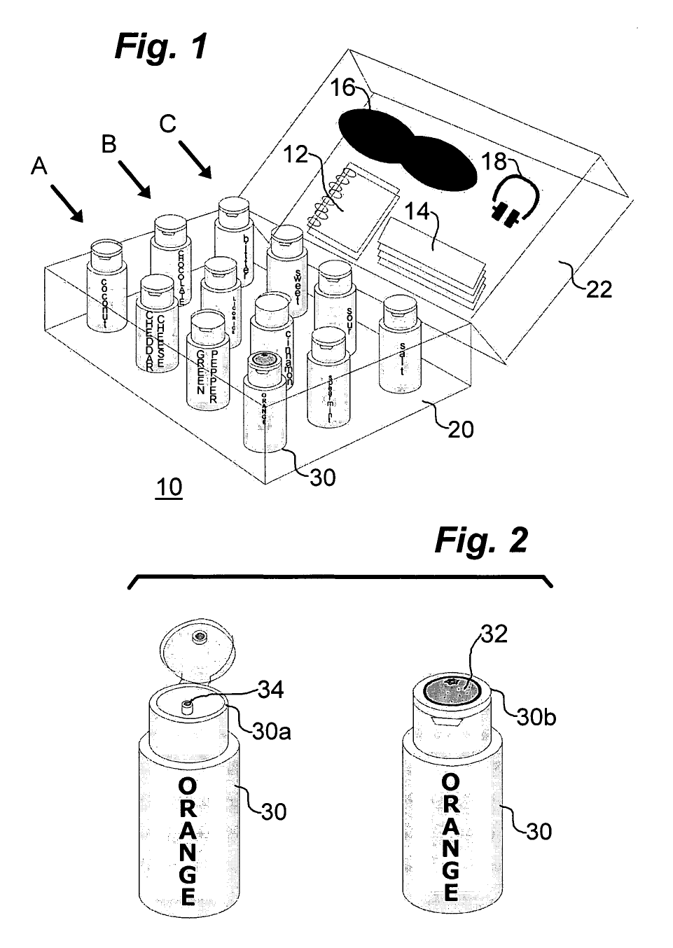

FIG. 1 is a simplified representation of a specific illustrative embodiment of the invention configured as a kit 10 having three rows A, B, and C, of four samples each. In this embodiment, rows A and B contain fragrances, and row C contains the basic tastes. There are additionally provided in this embodiment of kit 10 a plurality of fragrance cards 12, a supply of edible paper 14, and eye-mask 16, and a nose-clip 18. The kit is contained in an enclosure having a bottom contained portion 20 and a top portion 22 attached to each other by a hinge (not shown).

The kit contains four basic tastes, including salt (sodium chloride), sour (citric acid), sweet (sugar), and bitter (quinine sulfate), as identified on each sample of row C. In addition, the present embodiment of kit 10 contains a variable number of scents. As depicted in this figure, there are included in the present embodiment of the kit eight scents from various edible categories and with varying hedonic quality. They are, in row A: orange, green pepper, cheddar cheese, and coconut, and in row B, spearmint, cinnamon, licorice, chocolate. Tastes and smells are also referred to as “samples.” All samples in the present specific illustrative embodiment of the invention are liquid (not shown) and contained in 1 oz/30 ml white HDPE (high density polyethylene) cylindrical bottles, with screw-on flip-top lids of the same material with 0.031 inch (2 mm) orifice reducers. HDPE containers are typically used for milk and juice products and are fully recyclable. The samples are in clear liquid form dissolved in food grade propylene glycol (not shown). Propylene glycol is widely used in flavorings and was specifically chosen as the carrier medium because it minimizes stains from spillage, as compared to oil. All samples are food grade edible, kosher, and non-allergenic; and are either categorized as GRAS (Generally Regarded as Safe), have FEMA numbers, or both. The fragrances are calorie free and the tastes, with the exception of the negligible calories from sweet (sugar), are calorie free.

In a specific illustrative embodiment of the invention, the fragrances and tastes are supplied by Flavor Sciences, Inc. a privately owned company located in Stamford, Conn., that has been in operation since 1969, the principal activity of which is the creation, application, and production of flavoring materials. Other sources of tastes and flavors are Flavor and Fragrance Specialties, 3 Industrial Way, Mahwah, N.J. 07430; Mastertaste, Inc., 100 Hollister Road, Teterboro, N.J. 07608; and Flavor dynamics, Inc., 640 Montrose Avenue, South Plainfield, N.J. 07080.

Although no components to which there are already known allergic reactions, such as peanuts, are used, allergic reactions may occur in rare individuals. As such warning labels to this effect, as well as instructions not to apply into the eye are affixed to packaging. To dispense the sample, the child holds the bottle upside down and gives it an up-down shake. Only one small droplet of liquid (not shown) comes out from orifice 34 (FIG. 2) at a time. Sample names are displayed vertically in large, black lettering on the side of the bottles. A colored picture of the sample source is on the bottle lid, as will be discussed in connection with FIG. 2.

Tastes will only be identified by name. The twelve sample bottles are contained in enclosure 20, which in one embodiment of the invention is a hard cardboard/plastic/metal. The prototype case is monochrome (color may vary) of dimensions measuring 8 inches wide, and 4 inches deep, with a snap closing lid 22 hinged thereto. Inside the case are twelve wells (not specifically designated) for the scent and taste dispensers, which as stated are arranged in three rows, A, B, and C, two rows for scents and one row for tastes, of four samples each. In a practical embodiment, the inside lid of the case will map out with pictures where each scent and taste belongs in the array and the slot where the sample is housed can be designated with its visual icon (not shown). This reinforces consistent replacement in the designated well, so as to minimize cross-scent contamination.

FIG. 2 is an enlarged representation of a fragrance sample container 30 as shown in FIG. 1, in open 30a and closed 30b conditions. An orifice 34 is shown on the top of sample container 30 in open condition 30a, and an icon 32 is shown on the top of the sample container in closed condition 30b.

Referring once again to FIG. 1, included in kit 10 is a taste-free edible paper 14, a blindfold 16 and a nose-clip 18. The edible paper (also known as “rice paper” or “wafer paper”) used in this specific illustrative embodiment of the invention is made from potato starch, water, and vegetable oil in Holland by Primus Ouwelfabriek B.V. and prepared according to HACCP standards, a worldwide standard for the ultimate in sanitation. Edible paper 14 is configured in 3 cm ×7 cm stripes and wrapped in cellophane (not specifically designated) for packaging. The edible paper is intended to be the substrate where the sample liquids are applied for tasting and where mixing of the samples takes place. A thin cloth blindfold 16 is used for guessing what sample is being presented, and a swimmers style plastic nose clip 18 is used to demonstrate how flavor is dependent on retronasal olfaction.

Kit 10 is also shown to contain 12 cards 12 with pictures and information about the sample fragrances and tastes. For example: “True cinnamon comes from the stem bark of the cinnamomum zeylanicum tree, which is a member of the laurel family. Native to South Asia, it is a popular spice in Indian and Arabic savory dishes. In the West it is used mostly for desserts and confections.” A pamphlet containing basic information about the senses of smell and taste (not shown) is included as well as suggested instructions for classroom demonstrations and usage, in certain embodiments of the invention. In a larger system context, a website (not shown) is provided where further information and other materials, including how to obtain theme kits (e.g., all fruits) and other sample fragrances are available.

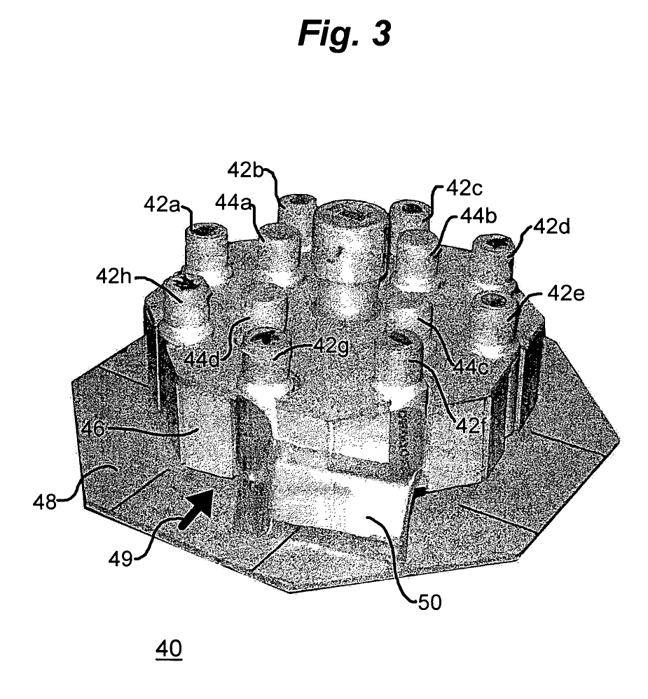

FIG. 3 is a perspective representation of a further specific illustrative embodiment of the invention, configured as a kit having a plurality of samples in a rotatable game arrangement 40. As shown in this figure, rotatable game arrangement 40 is in the form of a prototype embodiment of an educational toy game that will directly introduce children (not shown) to the fascinating world of smell, taste, and flavor.

By playing and experimenting with, and exploring, flavors 42a to 42h and tastes 44a to 44d in rotatable game arrangement 40, children will learn systematic names and concepts for specific flavors, scents and tastes; they will creatively and deductively discover how flavors are made; they will learn the differences between the senses of smell and taste; and they will be able to create novel flavor concoctions. In addition to amusing themselves, their friends and their family, the present invention makes children more familiar with novel flavor sensations. This exposure will enable children to overcome food neophobias and promote acceptance of a more varied diet for long-term health.

Rotatable game arrangement 40, as noted, is shown to be a prototype of a game system that, in this specific illustrative embodiment of the invention, is configured for persons of all ages, and for one or to multiple players. As shown, the specific embodiment of rotatable game arrangement 40 is provided with eight edible flavors, 42a to 42h and four edible tastes 44a to 44d. The eight edible flavors and four edible tastes are mounted in a carrier 46 that is itself mounted on a base 48. In addition, there is provided a package 50 that in this embodiment contains edible paper, playing cards, an eye-mask, and a nose-clip (not specifically designated).

Carrier 46 is rotatable on base 48 to permit the carrier to be spun by the player (not shown), and a pointer 49 on base 48 designates where each spin stops. Players will mix flavors and tastants on strips of the edible paper to sample them. The eye mask (not shown) will be used for flavor guessing and the nose-clip (not shown) to demonstrate that flavor is dependent on smell.

The eight flavors in the prototype rotatable game arrangement 40 are: cinnamon 42a, orange 42b, licorice 42c, chocolate 42d, green-pepper 42e, cheddar cheese 42f, spearmint 42g, and coconut 42h. The flavors created for embodiments of the present invention will be the most prototypical exemplars of their source.

In this specific illustrative embodiment of the invention, the four tastes are: salt 44a, sour 44b, sweet 44c, bitter 44d. In the practice of the invention, all taste samples are liquid and cloudy white monochromatic, or clear, so that visual cues can not be used for guessing or establishing expectations. The liquids are configured to have a viscosity that is similar to that of oil, or may be slightly gelled, so as minimize dripping and to support mixing.

The samples are shown in this figure to be contained in 1 oz/30 ml white LDPE (low density polyethylene) cylindrical bottles, with screw-on flip-top lids formed of the same material, with 0.031 inch (2 mm) orifice reducers (not shown). All samples are food grade edible, kosher, and non-allergenic; and will either be categorized as GRAS, or have FEMA numbers, or both. It is not necessary that in the practice of the invention the samples be comprised of natural ingredients. The fragrances and the tastes will have none or negligible caloric content. Containers will be labeled with stickers (not specifically designated) on their side and/or top indicating the identity of sample flavor or tastant by both picture and word .

A production version (not shown) of the present invention may be provided with a warning label (not shown) on the packaging that advises that ingestion of large quantities of liquid could produce an upset stomach. The warning label may also recommend against usage of the product if the player is known to have chemical allergies, and against permitting the liquids to contact the eyes.

There are several game forms that can be played within the scope of the present invention. A straightforward game will have children (not shown) spin the top of the sample wheel and perform the task indicated on the board at that spot where the wheel-pointer lands (e.g., “mix orange with sweet and sour”). Board instructions may also be in pictorial rather than verbal.

Another game form includes using playing cards (not shown) as well as the board instructions. For example, in one specific illustrative embodiment of the invention, the wheel-pointer will stop at a board space indicating that the child has to pick a playing card. Playing cards will be printed with instructions such as: “Put on the blindfold and have the person to your left mix two flavors with one taste.” The blindfolded player will then be required to guess the composition of the mixture, and he or she will receive a score of between 0 and 3. A different playing card would instruct the player to put on the nose-clip and blindfold, the player across from the blindfolded player will mix a flavor. The blindfolded player will then guess at what the mixture contains. The blindfolded player is then instructed to remove the nose-clip and try again to guess the composition. For each attempt, the blindfolded player is awarded five points for guessing correctly, and will receive additional points for correctly identifying the components, as appropriate. On yet another playing card, the selected player is instructed to tell the person on his or her right to put on the blind-fold. The selected player then mixes one taste and two flavors, and the player to the right with the blindfold will guess at the composition. One point is awarded for every correct answer, no points if they don't know, and one point is taken away for each mistake.

Playing cards may also simply illustrate pictures of two or more flavor and/or taste sources with instructions to mix the flavors/tastes illustrated and indicate the result. Cards are included in some embodiments of the kit-game that give detailed and easy to understand descriptions with pictures of the flavor sources. For example:

“Spearmint is a flavor in the mint category that is used in candy, chewing gum and sauces. It is a leafy plant with pale purple or mauve flowers that bloom from July to September. It is native to the Mediterranean region but also grows wild and is cultivated in temperate climates over most of the world.”

The present invention is adaptable to adult variations, such as with the use of wines (not shown). Any kit or game that contains smells, tastes, and flavors can be used for teaching or play, including animal training. For example, kits can be configured to train dogs (not shown) to recognize specific contraband scents.

Although the invention has been described in terms of specific embodiments and applications, persons skilled in the art may, in light of this teaching, generate additional embodiments without exceeding the scope or departing from the spirit of the invention described herein. Accordingly, it is to be understood that the drawing and description in this disclosure are proffered to facilitate comprehension of the invention, and should not be construed to limit the scope thereof.

1. A method of educating a living being to correlate taste and olfactory perception to an objective element, the method comprising the steps of:
providing a plurality of known taste and flavor ingestibles;
combining selectable ones of the flavor ingestibles and taste ingestibles to form a combination ingestible;
directing the living being to ingest the combination ingestible; and
training the living being to identify the combination ingestible by reference to the objective element.

2. The method of claim 1, wherein the living being is a human being and the objective element is a word that characterizes the perception resulting from the ingestion of the combination ingestible.

3. The method of claim 2, wherein the combination ingestible comprises a combination
exclusively of a plurality of the known taste ingestibles.

4. The method of claim 2, wherein the combination ingestible comprises a combination exclusively of a plurality of the known flavor ingestibles.

5. The method of claim 1, wherein the living being is an animal and the objective element is a predetermined scent.

6. The method of claim 5, wherein the predetermined scent is contraband scent.

7. The method of claim 1, wherein the plurality of known taste ingestibles is selected from the group consisting of salt, sour, sweet, bitter, and a combination thereof.

8. The method of claim 1, wherein the plurality of known flavor ingestibles is selected from the group consisting of cinnamon, orange, licorice, chocolate, green-pepper, cheddar cheese, spearmint, coconut, and a combination thereof.

9. The method of claim 1, wherein the method of educating a living being to correlate taste and olfactory perception to an objective element is configured as a guessing game.

10. The method of claim 9, wherein the plurality of known taste ingestibles and the plurality of known flavor ingestibles are configured to be unidentifiable other than by taste or smell.

11. The method of claim 9, wherein the plurality of known taste ingestibles and the plurality of known flavor ingestibles are in a form selected from the croup consisting of gels and liquids.

12. A method of playing a game, the method comprising the steps of:
combining selectable ones of a plurality of ingestibles to produce a combination ingestible; ingesting the combination ingestible; and
guessing a word that characterizes the perception resulting from said step of ingesting.

13. The method of claim 12, wherein said step of combining selectable ones of a plurality of ingestibles comprises the step of combining selectable ones of a plurality of known taste ingestibles.

14. The method of claim 13, wherein the known taste ingestibles are selected from the group consisting of salt, sour, sweet, bitter and a combination thereof.

15. The method of claim 12, wherein said step of combining selectable ones of a plurality of ingestibles comprises the step of combining selectable ones of a plurality of known flavor ingestibles.

16. The method of claim 15, wherein the known flavor ingestibles are selected from the group consisting of cinnamon, orange, licorice, chocolate, green-pepper, cheddar cheese, spearmint, coconut and a combination thereof.

17. The method of claim 12, wherein said step of combining selectable ones of a plurality of ingestibles comprises the step of combining selectable ones of a plurality of known taste ingestibles and a plurality of known flavor ingestibles.

18. A game kit comprising:
a plurality of known taste ingestibles;
a plurality of known flavor ingestibles; and
an edible substrate for mixing selectable ones of the known taste ingestibles and the known flavor ingestibles.

19. The game kit of claim 18, wherein there is further provided a rotatable carrier for supporting said plurality of known taste ingestibles and said plurality of known flavor ingestibles rotatively.

20. The game kit of claim 18, wherein there is further provided
a blindfold.

21. The game kit of claim 18 wherein there are further provided a nose clip.

22. The game kit of claim 18 wherein there is further provided a plurality of selectable cards containing instructions for a player.
